(19) United States
(12) Patent Application Publication
(10) Pub. No.: US 2015/0359447 A1
(43) Pub. Date: Dec. 17, 2015

(54) ELECTRONIC DEVICE, METHOD, AND COMPUTER PROGRAM PRODUCT

(71) Applicant: KABUSHIKI KAISHA TOSHIBA, Tokyo (JP)

(72) Inventors: Yasuhiro KANISHIMA, Suginami Tokyo (JP); Takashi SUDO, Fuchu Tokyo (JP)

(21) Appl. No.: 14/662,038

(22) Filed: Mar. 18, 2015

Related U.S. Application Data
(60) Provisional application No. 62/013,214, filed on Jun. 17, 2014.

(51) Int. Cl. A61B 5/024; A61B 5/00; G06F 21/31

(52) U.S. Cl. CPC A61B 5/02438; G06F 21/31; A61B 5/6824; A61B 5/7235; A61B 5/7221

(57) ABSTRACT

According to an embodiment, for example, an electronic device wearable on a body, the electronic device including: a first sensor to emit light to a body and to output a signal corresponding to a received amount of light that has passed through a body or has been reflected by a body; and a circuitry to determine whether the electronic device is on the body, by using amplitude and frequency information corresponding to a pulse in the signal output from the first sensor.

### CROSS-REFERENCE TO RELATED APPLICATIONS

This application claims the benefit of U.S. Provisional Patent Application No. 62/013,214, filed Jun. 17, 2014.

### FIELD

The embodiment disclosed herein generally relates to an electronic device, a method, and a computer program product.

### BACKGROUND

Electronic devices such as wearable devices are known that constantly acquire biological information such as a pulse and autonomic nerve conditions of a living body by using a first sensor such as a photoelectric pulse wave sensor.

The conventional wearable devices, however, record abnormal biological information in some cases because the wearable devices acquire biological information even when they are not worn by users or they are worn in an unstable state (for example, in a state in which a user is in motion to wear a wearable device).

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention.

FIG. 1 is an example of a perspective view illustrating a schematic configuration of a wearable terminal according to an embodiment;

FIG. 2 is a block diagram illustrating an example of a hardware configuration of the wearable terminal according to the embodiment;

FIG. 3 is a block diagram illustrating an example of a specific configuration of a first sensor included in the wearable terminal according to the embodiment, and an example of a functional configuration implemented by a CPU by executing a biological information acquisition program;

FIG. 4 is an exemplary diagram illustrating determination processing of a worn state on the basis of a sensor output signal in the wearable terminal according to the embodiment;

FIG. 5 is a flowchart illustrating an example of the procedure in the wearable terminal according to the embodiment for validating or invalidating biological information, and for controlling light emission from the first sensor;

FIG. 6 is a diagram illustrating an example of a change in signal levels of a sensor output signal output from the first sensor of the wearable terminal according to the embodiment;

FIG. 7 is a diagram illustrating an example of a frequency of a sensor output signal when the wearable terminal according to the embodiment is in a not-worn state; and

FIG. 8 is a diagram illustrating an example of a frequency of a sensor output signal when the wearable terminal according to the embodiment is in a worn state.

### DETAILED DESCRIPTION

In general, according to an embodiment, an electronic device wearable on a body, the electronic device comprising: a first sensor to emit light to a body and to output a signal corresponding to a received amount of light that has passed through a body or has been reflected by a body; and a circuitry to determine whether the electronic device is on the body, by using amplitude and frequency information corresponding to a pulse in the signal output from the first sensor.

The following describes a case in which the electronic device according to the present embodiment is applied to a wearable terminal that can be worn on a body (a human body in the present embodiment). Specifically, the following describes a case in which the electronic device according to the present embodiment is applied to a wearable terminal having a shape of a wristwatch that a user can constantly wear on an arm (such as a wrist).

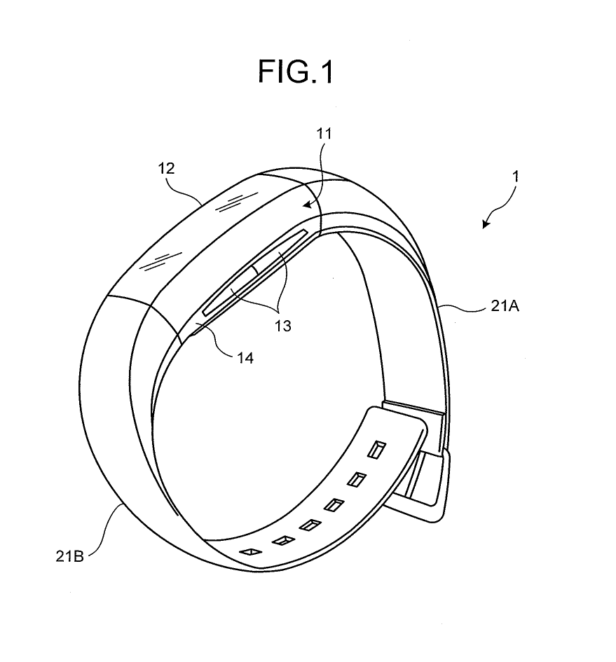

First, described is a schematic configuration of a wearable terminal 1 that is an example of the electronic device according to the present embodiment with reference to FIG. 1. FIG. 1 is an example of a perspective view illustrating the schematic configuration of the wearable terminal according to the present embodiment.

As illustrated in FIG. 1, the wearable terminal 1 according to the present embodiment includes a main body 11 including a thin housing 14 that is an example of a housing that can be worn on a body. The housing 14 stores various electronic parts therein. As illustrated in FIG. 1, the top surface of the main body 11 is provided with a display 12 that is a display unit configured by, for example, a liquid crystal display (LCD) and capable of displaying various types of information. The display 12 may be a touch panel display that can detect a contact position of a touch operation on the display 12. As illustrated in FIG. 1, a side of the main body 11 is provided with operating buttons 13 with which various types of operations can be input to the wearable terminal 1

As illustrated in FIG. 1, the wearable terminal 1 according to the present embodiment includes belts 21A and 21B with which the housing 14 can be worn on the body (an arm in the present embodiment). The belts 21A and 21B are composed of a flexible material.

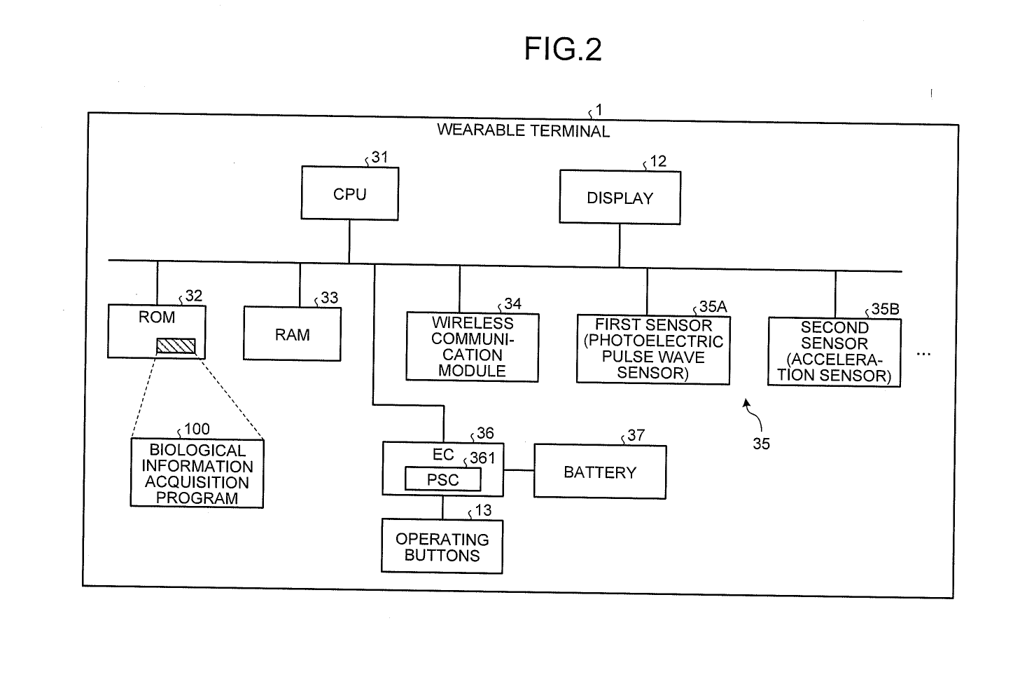

Described next is a hardware configuration of the wearable terminal 1 according to the present embodiment with reference to FIG. 2. FIG. 2 is a block diagram illustrating an example of the hardware configuration of the wearable terminal according to the embodiment.

As illustrated in FIG. 2, the wearable terminal 1 according to the present embodiment includes a central processing unit (CPU) 31, a read only memory (ROM) 32, a random access memory (RAM) 33, a wireless communication module 34, a plurality of sensors 35, an embedded controller (EC) 36, and a battery 37 in addition to the display 12 and the operating buttons 13 described above with reference to FIG. 1.

The CPU 31 is a processor that controls each module included in the wearable terminal 1. The CPU 31 uses the RAM 33 as a working area to execute various computer programs stored in the ROM 32. The various computer programs stored in the ROM 32 include a biological information acquisition program 100 for executing processing for acquiring biological information on the body to which the wearable terminal 1 is worn.

Specifically, the biological information acquisition program 100 is a computer program that executes processing for acquiring biological information such as a pulse of a user who wears the wearable terminal 1 or activity condition of the user's autonomic nerve on the basis of a signal output from a first sensor 35A to be described later.

The wireless communication module 34 can perform wireless communication in accordance with standards such as the IEEE 802.11g standards. The sensors 35 include various sensors such as the first sensor 35A (a photoelectric pulse wave sensor), a second sensor 35B (an acceleration sensor), an angular velocity sensor, a geomagnetism sensor, a temperature sensor, a humidity sensor, and an illuminance sensor.

The first sensor 35A is provided in the housing 14. The first sensor 35A is a photoelectric pulse wave sensor configured to emit light to a body and to output a signal corresponding to a received amount of light that has passed through the body or has been reflected on the body. In the present embodiment, the first sensor 35A is configured by, for example, a reflective photoelectric sensor. By using an action that hemoglobin in blood absorbs light, the first sensor 35A receives light emitted from the first sensor 35A to a blood vessel and reflected back thereto, and outputs a signal corresponding to the received amount of the reflected light.

Alternatively, the first sensor 35A may be configured by a through-beam photoelectric sensor. The first sensor 35A may be configured to output a signal corresponding to the received amount of light that has passed through the blood vessel. As blood in a blood vessel flows faster, hemoglobin in blood absorbs more light. In this case, both reflective photoelectric sensor and through-beam photoelectric sensor receive a smaller amount of light, that is, the reflective photoelectric sensor receives a smaller amount of light emitted to a blood vessel and reflected back to the sensor, and the through-beam photoelectric sensor receives a smaller amount of light that has passed through the blood vessel.

The second sensor 35B is configured to detect at least movement of the wearable terminal 1. In the present embodiment, the second sensor 35B is configured by, for example, a three-axis acceleration sensor and is provided in the housing 14. The second sensor 35B detects acceleration of the wearable terminal 1 as the movement of the wearable terminal 1.

The EC 36 is a one-chip microcomputer including a power supply controller (PSC) 361 that controls electric power supply from the battery 37 to the various modules of the wearable terminal 1. The EC 36 has a function of acquiring various instructions input by a user by using the operating buttons 13.

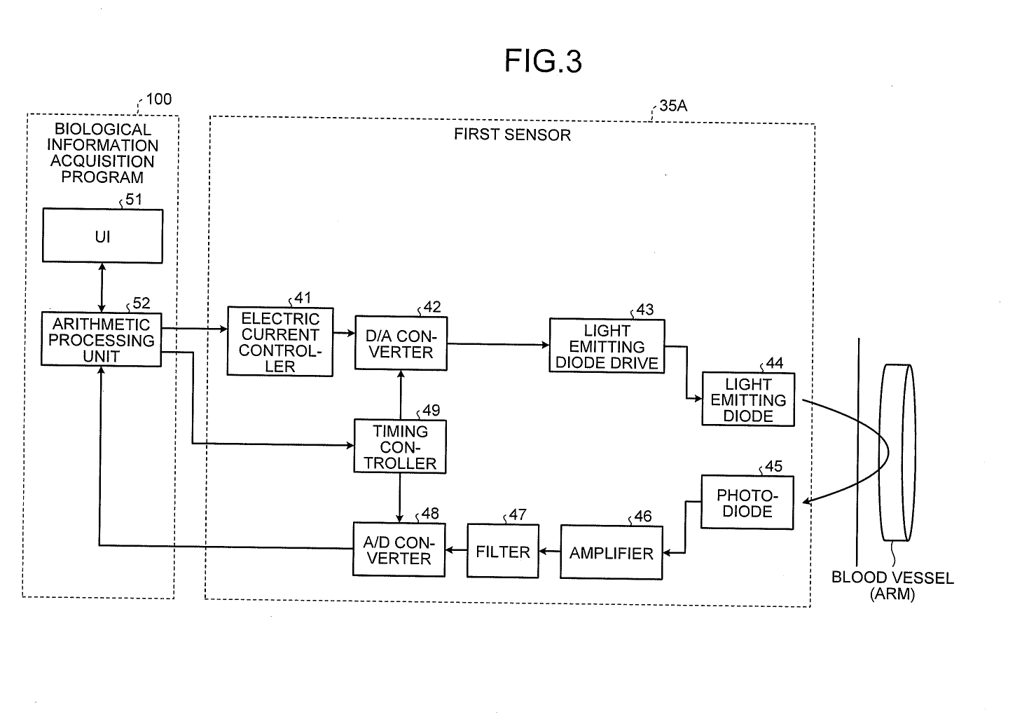

Described next is a specific configuration of the first sensor 35A included in the wearable terminal 1 according to the present embodiment, and a functional configuration implemented by the CPU 31 by executing the biological information acquisition program 100 with reference to FIG. 3. FIG. 3 is a block diagram illustrating an example of the specific configuration of the first sensor included in the wearable terminal according to the embodiment, and an example of the functional configuration implemented by the CPU by executing the biological information acquisition program.

First, described is a specific configuration of the first sensor 35A. In the present embodiment, the first sensor 35A includes, as illustrated in FIG. 3, an electric current controller 41, a D/A converter 42, a light emitting diode drive 43, a light emitting diode 44, a photodiode 45, an amplifier 46, a filter 47, an A/D converter 48, and a timing controller 49.

The light emitting diode 44 is a light emitting element configured to be able to emit light to a blood vessel. The photodiode 45 is a light receiving element configured to be able to receive light emitted from the light emitting diode 44 to a blood vessel and reflected back to the photodiode 45 (or light emitted from the light emitting diode 44 to a blood vessel and passing through blood in the blood vessel). The photodiode 45 outputs analog electric current corresponding to the received amount of light as a signal (hereinafter referred to as a sensor output signal) corresponding to the received amount of light.

The electric current controller 41 sends electric current from the battery 37 to the light emitting diode 44. The D/A converter 42 converts (D/A-converts) digital electric current sent from the electric current controller 41 into analog electric current, and outputs it. The light emitting diode drive 43 sends the analog electric current output from the D/A converter 42 to the light emitting diode 44 to illuminate the light emitting diode 44.

The amplifier 46 amplifies a sensor output signal output from the photodiode 45 by receiving light of the photodiode 45. The filter 47 removes noise from the sensor output signal amplified by the amplifier 46. The A/D converter 48 converts (A/D-converts) the sensor output signal from which noise is removed into a digital sensor output signal, and outputs it. The timing controller 49 controls timing of D/A conversion by the D/A converter 42, and timing of A/D conversion by the A/D converter 48.

Described next is a functional configuration implemented by the CPU 31 by executing the biological information acquisition program 100. In the present embodiment, the CPU 31 reads the biological information acquisition program from the ROM 32 and executes it to load a user interface (UI) 51 and an arithmetic processing unit 52 on the RAM 33. That is how the CPU 31 generates the UI 51 and the arithmetic processing unit 52 on the RAM 32.

The UI 51 outputs, to the arithmetic processing unit 52, various instructions (such as an instruction to turn on or turn off the power of the wearable terminal 1) input from the operating buttons 13. The arithmetic processing unit 52 controls the wearable terminal 1 in accordance with the instructions output from the UI 51.

The arithmetic processing unit 52 acquires biological information on the basis of a sensor output signal output from the first sensor 35A. Although, in the present embodiment, the wearable terminal 1 executes acquiring biological information on the basis of a sensor output signal, the embodiment is not limited to this. The wearable terminal 1 may output a sensor output signal to an external device, and the external device may execute acquiring biological information on the basis of the sensor output signal.

In the present embodiment, the arithmetic processing unit 52 (an example of the processor) is configured to determine whether the wearable terminal 1 is worn by use of whether a sensor output signal output from the first sensor 35A contains a vibration component (a characteristic of amplitude of the sensor output signal and a frequency of the sensor output signal in the present embodiment) of a frequency corresponding to a pulse. The arithmetic processing unit 52 determines whether the wearable terminal 1 (the housing 14 in the present embodiment) is in a certain worn state on the basis of a characteristic (such as the average, median, or standard deviation of signal levels) of signal levels (signal intensity) that are levels (amplitude) of a sensor output signal output from the first sensor 35A, and a frequency of the sensor output signal. The certain worn state is a state in which a user wears the housing 14. In other words, the certain worn state is a state other than not-worn states, that is, other than states in which a user is not wearing the housing 14 or in which the user is in motion to wear the housing 14.

When determining that the housing 14 is not in the certain worn state, the arithmetic processing unit 52 invalidates biological information acquired on the basis of a sensor output signal output from the first sensor 35A. Thus, the wearable terminal 1 according to the present embodiment invalidates biological information acquired when the housing 14 is in a not-worn state. This enables the wearable terminal 1 to avoid recording abnormal biological information acquired when the housing 14 is in a not-worn state.

The arithmetic processing unit 52 invalidates biological information by making biological information unacquirable on the basis of a sensor output signal output from the first sensor 35A when the housing 14 is in a not-worn state. In the present embodiment, the arithmetic processing unit 52 invalidates biological information by prohibiting the first sensor 35A from outputting a sensor output signal, or prohibiting itself from acquiring biological information on the basis of a sensor output signal.

In the electronic device such as the wearable terminal 1, the first sensor 35A consumes a large proportion of electric power relative to the entire power consumption of the wearable terminal 1. In the present embodiment, the arithmetic processing unit 52 controls the first sensor 35A to emit light depending on a state of use of the wearable terminal 1 (specifically, whether the wearable terminal 1 is worn by a user). Specifically, when the arithmetic processing unit 52 determines that the housing 14 is not in the certain worn state on the basis of a characteristic of signal levels of a sensor output signal output from the first sensor 35A and a frequency of the sensor output signal, the arithmetic processing unit 52 controls the first sensor 35A to stop emitting light. Thus, when the housing 14 is in a not-worn state, the first sensor 35A does not emit light, thereby reducing power consumption of the wearable terminal 1.

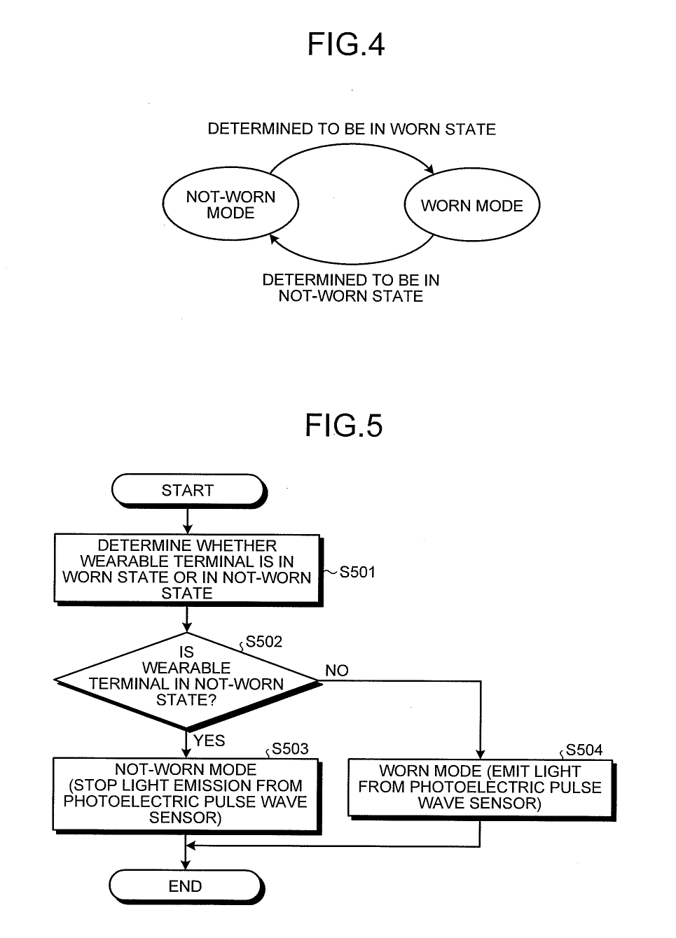

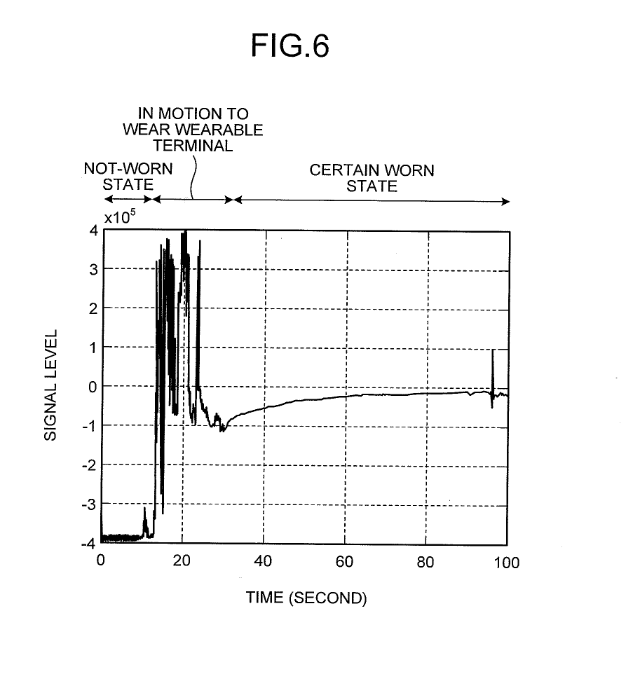

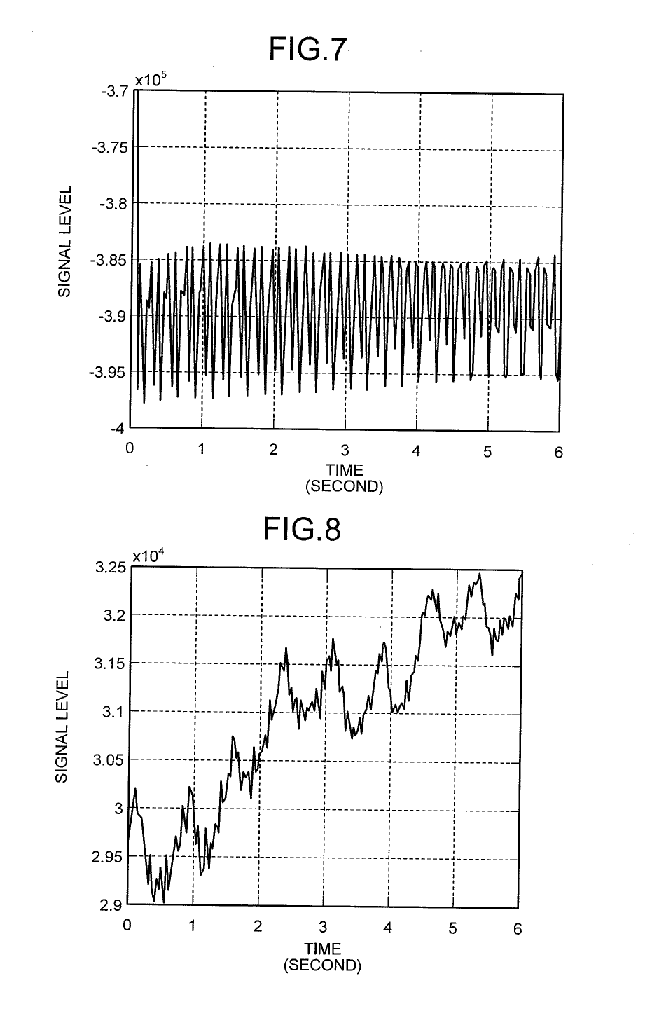

Described next are processing for validating or invalidating biological information in the wearable terminal 1 according to the present embodiment, and processing for controlling light emission from the first sensor 35A with reference to FIGS. 4 to 8. FIG. 4 is an exemplary diagram illustrating determination processing of a worn state on the basis of a sensor output signal in the wearable terminal according to the embodiment. FIG. 5 is a flowchart illustrating an example of the procedure in the wearable terminal according to the embodiment for validating or invalidating biological information, and for controlling light emission from the first sensor. FIG. 6 is a diagram illustrating an example of a change in signal levels of a sensor output signal output from the first sensor of the wearable terminal according to the embodiment. FIG. 7 is a diagram illustrating an example of a frequency of a sensor output signal when the wearable terminal according to the embodiment is in a not-worn state. FIG. 8 is a diagram illustrating an example of a frequency of a sensor output signal when the wearable terminal according to the embodiment is in a worn state.

When the UI 51 outputs an instruction to turn on the power of the wearable terminal 1, the PSC 361 starts sending electric power from the battery 37 to the various modules of the wearable terminal 1 such as the first sensor 35A.

When power is supplied from the battery 37 to the first sensor 35A and the light emitting diode 44 starts emitting light, the arithmetic processing unit 52 determines whether the housing 14 is in the certain worn state or in a not-worn state by use of whether a sensor output signal output from the A/D converter 48 contains a vibration component of a frequency corresponding to a pulse (S501).

The first sensor 35A has a characteristic which amplitude (that is, signal levels) of a sensor output signal output from the first sensor 35A varies greatly depending on whether an object exists that reflects light emitted from the first sensor 35A.

In the present embodiment, when the average signal level that is an example of a characteristic of signal levels of a sensor output signal per certain unit time (for example, 20 seconds) is smaller than a certain signal level (for example, −1) (see the time period of the not-worn state illustrated in FIG. 6), and a frequency of the sensor output signal per certain unit time is not a certain frequency (see FIG. 7), the arithmetic processing unit 52 determines that the housing 14 is in a not-worn state. On the other hand, when the average signal level of a sensor output signal per certain unit time is the same as or larger than a certain signal level (see the time period of the worn state illustrated in FIG. 6), and a frequency of the sensor output signal per certain unit time is a certain frequency (see FIG. 8), the arithmetic processing unit 52 determines that the housing 14 is in a worn state.

The certain signal level is a signal level of the sensor output signal output from the first sensor 35A when the housing 14 is in a worn state in which the housing 14 is worn on a body of a user. The certain frequency is a frequency of the sensor output signal output from the first sensor 35A when the housing 14 is in a worn state. Specifically, the certain frequency is a frequency (see FIG. 8) corresponding to a pulse rate of a living body.

In the present embodiment, the arithmetic processing unit 52 determines whether the housing 14 is in the certain worn state on the basis of both average signal level that is an example of a characteristic of signal levels of a sensor output signal, and frequency of the sensor output signal. The arithmetic processing unit 52, however, may determine whether the housing 14 is in the certain worn state on the basis of at least one of a characteristic of signal levels of a sensor output signal and a frequency of the sensor output signal. Specifically, the arithmetic processing unit 52 determines that the housing 14 is in a worn state when the average signal level that is an example of a characteristic of signal levels of a sensor output signal is the same as or larger than a certain signal level irrespective of a frequency of the sensor output signal. The arithmetic processing unit 52 determines that the housing 14 is in a not-worn state when the average signal level that is an example of a characteristic of signal levels of a sensor output signal is smaller than a certain signal level irrespective of a frequency of the sensor output signal.

The arithmetic processing unit 52 determines that the housing 14 is in a worn state when the frequency of a sensor output signal is a certain frequency irrespective of a characteristic of signal levels of the sensor output signal. The arithmetic processing unit 52 determines that the housing 14 is in a not-worn state when the frequency of a sensor output signal is not a certain frequency irrespective of a characteristic of signal levels of the sensor output signal.

Even though the average signal level that is an example of a characteristic of signal levels of a sensor output signal per certain unit time is the same as or larger than a certain signal level, the arithmetic processing unit 52 may determine that the housing 14 is in a not-worn state (a state in which a user is in motion to wear the housing 14 as illustrated in FIG. 6) when the standard deviation of signal levels per certain unit time that is another example of a characteristic of signal levels of a sensor output signal is the same as or larger than a certain standard deviation. This enables the arithmetic processing unit 52 to determine that the housing 14 is in a not-worn state when a user is in motion to wear the housing 14, whereby the arithmetic processing unit 52 can determine whether the housing 14 is in a worn state with high accuracy.

The description returns to FIG. 5. When the arithmetic processing unit 52 determines that the housing 14 is in a not-worn state (Yes at S502), the arithmetic processing unit 52 causes the wearable terminal 1 to be set to a not-worn mode as illustrated in FIG. 4 (S503). In the present embodiment, the not-worn mode is a mode in which the arithmetic processing unit 52 invalidates biological information acquired on the basis of a sensor output signal and stops light emission from the first sensor 35A. Although, in the present embodiment, the arithmetic processing unit 52 invalidates biological information acquired on the basis of a sensor output signal and stops light emission from the first sensor 35A in the not-worn mode, the arithmetic processing unit 52 may at least invalidate biological information acquired on the basis of a sensor output signal in the not-worn mode. For example, in the not-worn mode, the arithmetic processing unit 52 may only invalidate biological information acquired on the basis of a sensor output signal without stopping light emission from the first sensor 35A.

When the arithmetic processing unit 52 determines that the housing 14 is in the certain worn state (No at S502), the arithmetic processing unit 52 causes the wearable terminal 1 to be set to a worn mode as illustrated in FIG. 4 (S504). In the present embodiment, the worn mode is a mode in which the arithmetic processing unit 52 permits acquisition of biological information on the basis of a sensor output signal and keeps light emission from the first sensor 35A.

According to the wearable terminal 1 according to the present embodiment, biological information is invalidated that is acquired when the housing 14 is in a not-worn state, thereby preventing recording of abnormal biological information acquired when the housing 14 is in a not-worn state.

Although, in the present embodiment, the arithmetic processing unit 52 determines whether the housing 14 is in the certain worn state only by use of whether a sensor output signal output from the first sensor 35A contains a vibration component of a frequency corresponding to a pulse, the embodiment is not limited to this. The arithmetic processing unit 52 may determine whether the housing 14 is in the certain worn state on the basis of a detection result of other sensors 35 (such as the second sensor 35B and the temperature sensor) in addition to the sensor output signal output from the first sensor 35A.

Specifically, when the arithmetic processing unit 52 determines that the housing 14 is not in the certain worn state on the basis of signal levels of a sensor output signal output from the first sensor 35A, and determines that body movement is present on the basis of a detection result of acceleration detected by the second sensor 35B, the arithmetic processing unit 52 redetermines whether the housing 14 is in the certain worn state on the basis of signal levels of the sensor output signal. Accordingly, even when the arithmetic processing unit 52 incorrectly determines that the housing 14 is not in the certain worn state on the basis of a sensor output signal, the arithmetic processing unit 52 redetermines whether the housing 14 is in the certain worn state on the basis of the sensor output signal, thereby improving accuracy in determining whether the housing 14 is in the certain worn state.

When the arithmetic processing unit 52 determines that the housing 14 is not in the certain worn state on the basis of a sensor output signal and determines that body movement is not present on the basis of a detection result of acceleration detected by the second sensor 35B, the arithmetic processing unit 52 may invalidate biological information acquired on the basis of the sensor output signal.

When the arithmetic processing unit 52 determines that the housing 14 is in the certain worn state on the basis of a sensor output signal, and determines that body movement is present on the basis of a detection result of acceleration detected by the second sensor 35B, the arithmetic processing unit 52 may permit acquisition of biological information on the basis of the sensor output signal.

When the arithmetic processing unit 52 determines that the housing 14 is in the certain worn state on the basis of signal levels of a sensor output signal, and determines that body movement is not present on the basis of a detection result of acceleration detected by the second sensor 35B, the arithmetic processing unit 52 may redetermine whether the housing 14 is in the certain worn state on the basis of the sensor output signal, or may invalidate biological information acquired on the basis of the sensor output signal.

When the arithmetic processing unit 52 determines that the housing 14 is not in the certain worn state on the basis of a sensor output signal output from the first sensor 35A, and determines that the user wears the housing 14 on the basis of a detection result of temperature detected by a temperature sensor 35, the arithmetic processing unit 52 redetermines whether the housing 14 is in the certain worn state on the basis of the sensor output signal. Accordingly, even when the arithmetic processing unit 52 incorrectly determines that the housing 14 is not in the certain worn state on the basis of a sensor output signal, the arithmetic processing unit 52 redetermines whether the housing 14 is in the certain worn state on the basis of the sensor output signal, thereby improving accuracy in determining whether the housing 14 is in the certain worn state.

When the arithmetic processing unit 52 determines that the housing 14 is not in the certain worn state on the basis of a sensor output signal, and determines that the housing 14 is not worn by a user on the basis of a detection result of temperature detected by the temperature sensor, the arithmetic processing unit 52 may invalidate biological information acquired on the basis of the sensor output signal.

When the arithmetic processing unit 52 determines that the housing 14 is in the certain worn state on the basis of a sensor output signal, and determines that the housing 14 is worn by a user on the basis of a detection result of temperature detected by the temperature sensor, the arithmetic processing unit 52 may permit acquisition of biological information on the basis of the sensor output signal.

When the arithmetic processing unit 52 determines that the housing 14 is in the certain worn state on the basis of a sensor output signal, and determines that the housing 14 is not worn by a user on the basis of a detection result of temperature detected by the temperature sensor, the arithmetic processing unit 52 may redetermine whether the housing 14 is in the certain worn state on the basis of the sensor output signal, or may invalidate biological information acquired on the basis of the sensor output signal.

The arithmetic processing unit 52 performs authentication processing on a user who wears the housing 14. Specifically, the arithmetic processing unit 52 executes the authentication processing by using information (such as a password) for authenticating the user input by using the operating buttons 13. When the arithmetic processing unit 52 successfully authenticates the user through the authentication processing, the arithmetic processing unit 52 permits the user to operate the wearable terminal 1. When the arithmetic processing unit 52 fails to authenticate the user through the authentication processing, the arithmetic processing unit 52 prohibits the user from operating the wearable terminal 1. After the arithmetic processing unit 52 successfully authenticates the user through the authentication processing and permits the user to operate the wearable terminal 1, when the arithmetic processing unit 52 determines that the housing 14 is not in the certain worn state on the basis of a sensor output signal, the arithmetic processing unit 52 invalidates the result of the authentication processing.

Accordingly, even when the arithmetic processing unit 52 successfully authenticates a user through the authentication processing, the arithmetic processing unit 52 can make the wearable terminal 1 inoperative state when the housing 14 is not in the certain worn state. This can prevent users other than the user who performed the authentication processing from operating the wearable terminal 1 when the user left the wearable terminal 1 without wearing it after the authentication processing of the user succeeded.

Moreover, the various modules of the systems described herein can be implemented as software applications, hardware and/or software modules, or components on one or more computers, such as servers. While the various modules are illustrated separately, they may share some or all of the same underlying logic or code.

While certain embodiments have been described, these embodiments have been presented by way of example only, and are not intended to limit the scope of the invention. Indeed, the novel embodiments described herein may be embodied in a variety of other forms; furthermore, various omissions, substitutions and changes in the form of the embodiments described herein may be made without departing from the spirit of the invention. The accompanying claims and their equivalents are intended to cover such forms or modifications as would fall within the scope and spirit of the invention.

What is claimed is:

1. An electronic device wearable on a body, the electronic device comprising:
a first sensor to emit light to a body and to output a signal corresponding to a received amount of light that has passed through a body or has been reflected by a body; and
a circuitry to determine whether the electronic device is on the body, by using amplitude and frequency information corresponding to a pulse in the signal output from the first sensor.

2. The electronic device of claim 1, wherein
the circuitry comprises to cause the first sensor to stop emitting light when the circuitry determines that the electronic device is not on the body.

3. The electronic device of claim 1, wherein
the circuitry further comprises to invalidate biological information acquired from the first sensor by using the signal output from the first sensor when the circuitry determines that the electronic device is not on the body.

4. The electronic device of claim 1, further comprising:
a second sensor to detect at least movement of the electronic device, wherein
when the circuitry determines that the electronic device is not on the body by using the signal output from the first sensor, and that the body wearing the electronic device is moving by using a signal output from the second sensor, the circuitry comprises to redetermine whether the electronic device is on the body by using the signal output from the first sensor.

5. The electronic device of claim 1, wherein
the circuitry further comprises to perform authentication of a user who wears the electronic device, wherein
after the authentication of the user succeeds and the circuitry permits the user to operate the electronic device, the circuitry comprises to invalidate the authentication result when the circuitry determines that the electronic device is not on the body.

6. A method of controlling an electronic device comprising:
receiving a signal output from a first sensor in an electronic device wearable on a body, the signal corresponding to a received amount of light that has passed through a body or has been reflected by a body; and
determining whether the electronic device is on the body by using amplitude and frequency information corresponding to a pulse in the received signal output from the first sensor.

7. The method of claim 6, further comprising,
causing the first sensor to stop emitting light, when the electronic device is determined not on the body.

8. The method of claim 6, further comprising,
invalidating biological information acquired by using the signal output from the first sensor, when the electronic device is determined not on the body.

9. The method of claim 6, further comprising
when the electronic device is determined not on the body by using the signal output from the first sensor, and the body wearing the electronic device is determined to be moving by using a signal output from a second sensor configured to detect at least movement of the electronic device,
redetermining whether the electronic device is on the body by using the signal output from the first sensor.

10. The method of claim 6, further comprising:
performing authentication of a user who wears the electronic device, wherein
after the authentication succeeds and operations on the electronic device are permitted to the user, invalidating the authentication result when the electronic device is determined not on the body.

11. A non-transitory computer-readable medium having a plurality of executable instructions configured to cause one or more computers to perform processing, the computer-readable medium comprising:
receiving a signal output from a first sensor in an electronic device wearable on a body, the signal corresponding to a received amount of light that has passed through a body or has been reflected by a body; and
determining whether the electronic device is on the body by using amplitude and frequency information corresponding to a pulse in the received signal output from the first sensor.

12. The computer-readable medium of claim 11, wherein
causing the first sensor to stop emitting light, when the electronic device is determined not on the body.

13. The computer-readable medium of claim 11, wherein
invalidating biological information acquired by using the signal output from the first sensor, when the electronic device is determined not on the body.

14. The computer-readable medium of claim 11, wherein,
when the electronic device is determined not on the body by using the signal output from the first sensor, and the body wearing the electronic device is determined to be moving by using a signal output from a second sensor configured to detect at least movement of the electronic device,
redetermining whether the electronic device is on the body by using the signal output from the first sensor.

15. The computer-readable medium of claim 11, further comprising:
performing authentication of a user who wears the electronic device, wherein
after the authentication succeeds and operations on the electronic device are permitted, invalidating the authentication result when the electronic device is determined not on the body.
